(19) United States
(12) Patent Application Publication
(10) Pub. No.: US 2005/0202394 A1
(43) Pub. Date: Sep. 15, 2005

(54) Organ arrest, protection, preservation and recovery

(75) Inventor: Geoffrey Phillip Dobson, Wulguru (AU)

Correspondence Address: VENABLE LLP, P.O. BOX 34385, WASHINGTON, DC, 20045-9998, US

(73) Assignee: Global Cardiac Solutions Pty. Ltd., Queensland (AU)

(21) Appl. No.: 10/518,733

(22) PCT Filed: Jun. 20, 2003
(86) PCT No.: PCT/AU03/00771

§ 371 Date: Dec. 21, 2004

(51) Int. Cl. A01N 1/00; A61K 31/655

(52) U.S. Cl. 435/1.1; 514/149

(57) ABSTRACT

The present invention relates to a composition for controlling viability of a tissue including a potassium channel opener or adenosine receptor agonist, a compound for inducing local anaesthesia and a compound for reducing the uptake of water by a cell in the tissue. The present invention also realtes to the use of the composition accordng to the invention for controlling viability of a tissue.

The present invention relates to a composition for use in controlling viability of a tissue, for example arrested myocardial tissue and to uses of the composition for controlling viability of tissue.

### BACKGROUND OF THE INVENTION

There are over 20,000 open-heart surgery operations each year in Australia, over 800,000 in the United States and about 1,000,000 in Europe. During these procedures the human heart may be arrested for 3 hrs, and a maximum of 4 hrs. Arrest is achieved by the application of a cardioplegia solution directly to the heart.

Cardioplegia solutions arrest the heart using high potassium concentrations (in excess of 15-20 mM), which include the widely used St Thomas No. 2 Hospital Solution containing 110 mM NaCl, 16 mM KCl, 16 mM MgCl₂, 1.2 mM CaCl₂ and 10 mM NaHCO₃ and has a pH of about 7.8. High potassium solutions usually lead to a membrane depolarisation from about −80 to −50 mV. Notwithstanding hyperkalemic solutions providing acceptable clinical outcomes, recent evidence suggests that progressive potassium induced depolarisation leads to ionic and metabolic imbalances that may be linked to myocardial stunning, ventricular arrhythmias, ischaemic injury, endothelial cell swelling, microvascular damage, cell death and loss of pump function during the reperfusion period. Infant hearts are even more prone to damage with cardioplegic arrest from high potassium than adult hearts. In some cases, high potassium induced ischemia may also result in smooth muscle and endothelial function.

In addition, ischaemic heart disease is the single leading cause of death in the US and industrialised nations. Nearly half of the heart attacks are fatal, and half of these occur within the first hour of experiencing symptoms and before the patient reaches the hospital to be treated. Ischaemia (literally “to hold back blood”) is usually defined as an imbalance between blood supply and demand to an organ or tissue and results in deficient oxygen, fuel or nutrient supply to cells. The most common cause of ischaemia is a narrowing of the artery or, in the extreme case, from a blood clot blocking the artery. In 90% of those cases where a blood clot is the cause, the blood clot is usually formed from rupture of an atherosclerotic plaque.

The response of a cell to ischaemia depends upon the time and extent of the deprivation of blood supply. A large percentage of deaths from cardiac ischaemia are due to ventricular fibrillation (VF) associated with profound metabolic, ionic and functional disturbances. Within seconds to minutes of coronary artery occlusion there is a shift from aerobic to anaerobic metabolism, a decrease in high-energy phosphates (phosphocreatine, ATP), glycogen loss, lactate accumulation, tissue acidosis, a rise in intracellular Na⁺ and Ca²⁺ and extracellular K⁺ as well as changes to the transmembrane potential and ventricular dysfunction. Restoration of coronary flow within 15 min can lead to full recovery. However, it can also stun the myocardium and coronary vasculature leading to potentially fatal arrhythmias. If ischaemia persists beyond 15 min, the deprived area of the heart will undergo a progressive loss of ATP, increased Na⁺ and Ca²⁺ influx, severe membrane injury, sarcoplasmic reticulum mitochondrial dysfunction, and the closing of gap junctions between cells thereby electrically isolating the damaged cells and eventually, cell death will occur.

While early reperfusion or restoration of the blood flow remains the most effective means of salvaging the myocardium and coronary vasculature from acute ischaemia, the sudden influx of oxygen paradoxically may lead to further necrosis, ventricular arrhythmias and death. The extent of reperfusion injury has been linked to a cascade of inflammatory reactions including the generation of cytokines, leukocytes, reactive oxygen species and free radicals.

Reperfusion of ischaemic myocardium and coronary vasculature is necessary to salvage tissue from eventual death. However, reperfusion after even brief periods of ischaemia is associated with pathologic changes that represent either an acceleration of processes initiated during ischaemia per se, or new pathophysiological changes that were initiated after reperfusion. The degree and extent of “reperfusion injury” can be influenced by inflammatory responses in the myocardium and coronary vasculature. Ischaemia-reperfusion prompts a release of oxygen free radicals, cytokines and other pro-inflammatory mediators that activate both the neutrophils and the coronary vascular endothelium. The inflammatory process can lead to endothelial dysfunction, microvascular collapse and blood flow defects, myocardial infarction and apoptosis. Pharmacologic anti-inflammatory therapies targeting specific steps have been shown to decrease infarct size and myocardial injury. Adenosine and nitric oxide are two compounds which have been observed to have beneficial effects against such neutrophil-mediated inflammation.

The applicant previously found that the heart can be better protected after arrest by using an effective concentration of the potassium channel opener adenosine and the local anaesthetic lignocaine to arrest and then preserve the heart (WO 00/56145). The potassium channel opener leads the cell to a hyperpolarised state, shortening the action potential and decreasing Ca²⁺ influx into the cell. This solution does not rely on high potassium concentration in order to arrest the tissue, reducing the risk of potassium induce injury to the tissue.

Although, this solution provides improved recovery of the arrested heart, this is only achieved for only relatively short periods, ie for periods up to 3-4 hrs. As stated above, a human heart is normally only arrested for up to 3 hrs during any surgical period, at a maximum of 4 hrs. Arrest for periods beyond 3 hrs, increases the likelihood of irreversible damage to the heart tissue resulting in a gradual cell death or infarction of the myocardial tissue. Accordingly, the longer the heart is arrested there is increasing cell death, which inturn reduces the capacity of the organ to fully recover and regain function when restored from the arrested state. Additionally, the heart tissue (which includes electrical cells, myocardial cells and cells of the coronary vasculature) begins to irreversibly become increasingly damaged when experiencing ischemia. Any period longer than 15 mins is potentially fatal until blood flow is restored.

Accordingly there is a need for a composition which enables the tissue to be arrested and/or preserved for longer periods to minimise cell death, ie beyond 3-4 hrs and preferably at a temperature greater than 4° C. Moreover, there is a need for a long-term preservation composition for tissues. This would be particularly advantageous, for example, for transplanting tissue or organs which have been removed from a first patient intended to be transplanted into a second patient (or recipient) where the second patient is located at a geographical distance from the first patient which may prevent using currently available cardioplegia or arrest solutions. Present solutions do not provide that a recipient be located more than 2-3 hrs travelling time from the location where a donor organ becomes available, thus limiting the donor population. A longer arrest and preservation period could also provide for additional window of time available in which the transplantation surgical procedure can be performed. Ischaemic damage to the organ during preservation is believed to be a significant factor in determining preservation times, and therefore the outcome of the transplant. Heart transplant statistics have shown the risk of death in the first year after the transplant operation doubles if the donor heart is stored from 1 to 5 hours, and triples with 7 hrs storage times. In addition, older hearts are significantly less tolerant of ischaemia than younger hearts. According to the 1997 World Transplant Statistics, a total of 44,142 organ transplants (including heart, heart/lung, liver, pancreas and kidney) were performed in the USA, Australia, Canada and Europe, of which—5171 were heart transplants. There is a desperate shortage of organs to keep up with this demand. One area receiving enormous attention in order to overcome organ shortage is to harvest organs from non-human animals and transplant them into humans. This is referred to as xenotransplantation, ie transplantation from one species to another, which could also benefit from a long term preservation solution.

There is also a need for a composition which enables the tissue to be protected for longer periods, ie beyond 4-6 hrs to minimise damage or infarction size to the tissue. This would be particularly advantageous where a tissue is naturally arrested, for example by heart attack. Such a solution could be provided to the tissue to preserve the tissue or organ until a time that its function can be restored.

There is also a need for a composition which also assists the tissue to recover faster after long-term arrest or preservation. A composition which provides better protection during arrest or preservation enables the tissue to recover to normal function more quickly.

### SUMMARY OF THE INVENTION

The present invention seeks to at least minimise one of the above limitations and/or address these needs.

In one aspect, the present invention provides a composition for controlling viability of a tissue including:
a potassium channel opener or adenosine receptor agonist;
a compound for inducing local anaesthesia; and
a compound for reducing the uptake of water by a cell in the tissue.

In another aspect, the invention provides a composition for controlling viability of a tissue. The composition includes a potassium channel opener or adenosine receptor agonist, a compound for inducing local anaesthesia and diazoxide.

In another aspect, the invention provides a composition for controlling viability of a tissue. The composition includes a potassium channel opener or adenosine receptor agonist, a compound for inducing local anaesthesia, and a compound for inhibiting transport of sodium and hydrogen ions across a plasma membrane of a cell in the tissue.

In another aspect, the invention provides a composition for controlling viability of a tissue. The composition includes a potassium channel opener or adenosine receptor agonist, a compound for inducing local anaesthesia and an antioxidant.

In another aspect, the invention provides a composition for controlling viability of a tissue. The composition includes a potassium channel opener or adenosine receptor agonist, a compound for inducing local anaesthesia, a source of magnesium in an amount for increasing the amount of magnesium in a cell in the tissue and a source of calcium in an amount for decreasing the amount of calcium within a cell in the tissue.

In another aspect, the invention provides a method of controlling the viability of a tissue. The method includes the step of contacting the tissue with a composition according to the invention.

In another aspect, the invention provides a method for arresting a tissue. The method includes the step of contacting the tissue with a composition according to the invention.

In another aspect, the invention provides a method for preserving a tissue. The method includes the step of contacting the tissue with a composition according to the invention.

In another aspect, the invention provides a method for protecting a tissue. The method includes the step of contacting the tissue with a composition according to the invention.

In another aspect, the invention provides a use of a composition according to the invention for the manufacture of a medicament for controlling the viability of a tissue.

### DETAILED DESCRIPTION OF THE INVENTION

The inventor has surprisingly found that the inclusion of a compound for reducing the uptake of water by a cell in a tissue with a potassium channel opener or adenosine receptor agonist and a compound for inducing local anaesthesia permits the viability of explanted tissue to be maintained for up to 15 hours. Specifically, as described herein, it has been observed that the viability, or in other words, the function of an explanted rat heart that had been arrested for 15 hours was controlled, or in other words, preserved by contact with a composition comprising sucrose, adenosine and lignocaine. This is a surprising result because it has been observed by some that adenosine and lignocaine permit the function of explanted or otherwise isolated tissue to be protected for no longer than about 4 to 5 hours. Accordingly, the use of a compound for reducing the uptake of water by a cell in a tissue together with a potassium channel opener or adenosine receptor agonist and a compound for inducing local anaesthesia is particularly advantageous for permitting preservation of explanted tissue intended for transplantation during long distance transport which may incur up to 15 hours, or for permitting preservation of isolated tissue, for example, by-passed organs such as cardiac tissue during lengthy surgical procedures.

Thus in one aspect, the invention provides a composition for controlling viability of a tissue including:
a potassium channel opener or adenosine receptor agonist;
a compound for inducing local anaesthesia; and
a compound for reducing the uptake of water by a cell in the tissue.

A compound for reducing the uptake of water by a cell in the tissue tends to control water shifts, ie, the shift of water between the extracellular and intracellular environments. Accordingly, these compounds are involved in the control or regulation of osmosis. One consequence is that a compound for reducing the uptake of water by a cell in the tissue reduces cell swelling that is associated with Oedema, such as Oedema that can occur during ischemic injury.

Compounds for reducing the uptake of water by a cell in a tissue are typically impermeants or receptor antagonists or agonists.

An impermeant according to the present invention may be selected from one or more of the group consisting of: sucrose, pentastarch, hydroxyethyl starch, raffinose, mannitol, gluconate, lactobionate, and colloids. Colloids include albumin, hetastarch, polyethylene glycol (PEG), Dextran 40 and Dextran 60.

Cell swelling can also result from an inflammatory response which may be important during organ retrieval, preservation and surgical grafting. Substance P, an important pro-inflammatory neuropeptide is known to lead to cell oedema and therefore antagonists of substance P may reduce cell swelling. Indeed antagonists of substance P, (-specific neurokinin-1) receptor (NK-1) have been shown to reduce inflammatory liver damage, i.e., oedema formation, neutrophil infiltration, hepatocyte apoptosis, and necrosis. Two such NK-1 antagonists include CP-96,345 or [(2S,3S)-cis-2-(diphenylmethyl)-N-((2-methoxyphenyl)methyl)-1-azabicyclo(2.2.2.)-octan-3-amine (CP-96,345)] and L-733,060 or [(2S,3S)₃-([3,5-bis(trifluoromethyl)phenyl]methoxy)-2-phenylpiperidine]. R116301 or [(2R-trans)-4-[1-[3,5-bis(trifluoromethyl)benzoyl]-2-(phenylmethyl)-4-piperidinyl]-N-(2,6-dimethylphenyl)-1-acetamide (S)-Hydroxybutanedioate] is another specific, active neurokinin-1 (NK(1)) receptor antagonist with subnanomolar affinity for the human NK(1) receptor (K(i): 0.45 nM) and over 200-fold selectivity toward NK(2) and NK(3) receptors. Antagonists of neurokinin receptors 2 (NK-2) that may also reduce cell swelling include SR48968 and NK-3 include SR142801 and SB-222200. Blockade of mitochondrial permeability transition and reducing the membrane potential of the inner mitochondrial membrane potential using cyclosporin A has also been shown to decrease ischemia-induced cell swelling in isolated brain slices. In addition glutamate-receptor antagonists (AP5/CNQX) and reactive oxygen species scavengers (ascorbate, Trolox(R), dimethylthiourea, tempol(R)) also showed reduction of cell swelling. Thus, the compound for reducing the uptake of water by a cell in a tissue can also be selected from any one of these compounds.

Preferably the compound for reducing the uptake of water by the cells in the tissue is sucrose. Sucrose reduces water shifts as an impermeant. Impermeant agents such as sucrose, lactobionate and raffinose are too large to enter the cells and hence remain in the extracellular spaces within the tissue and resulting osmotic forces prevent cell swelling that would otherwise damage the tissue, which would occur particularly during storage of the tissue.

Preferably, the concentration of the compound for reducing the uptake of water by the cells in the tissue is between about 5 to 500 mM. Typically this is an effective amount for reducing the uptake of water by the cells in the tissue. More preferably, the concentration of the compound for reducing the uptake of water by the cells in the tissue is between about 20 and 100 uM. Even more preferably the concentration of the compound for reducing the uptake of water by the cells in the tissue is about 70 mM.

The inventor has also found that the inclusion of diazoxide with a potassium channel opener or adenosine receptor agonist and a compound for inducing local anaesthesia permits the viability of explanted tissue to be maintained for up to 15 hours. This is a surprising result because it has been observed by some that adenosine and lignocaine permit the function of explanted or otherwise isolated tissue to be protected for no longer than about 4 to 5 hours.

Thus in another aspect, the invention provides a composition for controlling viability of a tissue including:
a potassium channel opener or adenosine receptor agonist;
a compound for inducing local anaesthesia; and
diazoxide.

Diazoxide is a potassium channel opener and in the present invention it is believed to preserve ion and volume regulation, oxidative phosphorylation and mitochondrial membrane integrity (appears concentration dependent). More recently, diazoxide has been shown to provide cardioprotection by reducing mitochondrial oxidant stress at reoxygenation. At present it is not known if the protective effects of potassium channel openers are associated with modulation of reactive oxygen species generation in mitochondria.

Preferably the concentration of the diazoxide is between about 1 to 200 uM. Typically this is as an effective amount of diazoxide. More preferably, the concentration of diazoxide is about 10 uM.

The inventor has also found that the inclusion of a compound for inhibiting transport of sodium and hydrogen ions across a plasma membrane of a cell in the tissue with a potassium channel opener or adenosine receptor agonist and a compound for inducing local anaesthesia permits the viability of explanted tissue to be maintained for up to 15 hours. This is a surprising result because it has been observed by some that adenosine and lignocaine permit the function of explanted or otherwise isolated tissue to be protected for no longer than about 4 to 5 hours.

Thus in another aspect, the invention provides a composition for controlling viability of a tissue including:
a potassium channel opener or adenosine receptor agonist;
a compound for inducing local anaesthesia; and
a compound for inhibiting transport of sodium and hydrogen ions across a plasma membrane of a cell in the tissue.

The compound for inhibiting transport of sodium and hydrogen across the membrane of the cell in the tissue is also referred to as a sodium hydrogen exchange inhibitor. The sodium hydrogen exchange inhibitor reduces sodium and calcium entering the cell.

Preferably the compound for inhibiting transport of sodium and hydrogen across the membrane of the cell in the tissue may be selected from one or more of the group consisting of Amiloride, EIPA(5-(N-entyl-N-isopropyl)-amiloride), cariporide (HOE-642), eniporide, Triamterene (2,4,7-triamino-6-phenylteride), EMD 84021, EMD 94309, EMD 96785, EMD 85131, HOE 694. B11 B-513 and T-162559 are other inhibitors of the isoform 1 of the Na⁺/H⁺ exchanger.

Preferably, the sodium hydrogen exchange inhibitor is Amiloride (N-amidino-3,5-diamino-6-chloropyrzine-2-carboximide hydrochloride dihydrate). Amiloride inhibits the sodium proton exchanger (Na⁺/H⁺ exchanger also often abbreviated NHE-1) and reduces calcium entering the cell. During ischemia excess cell protons (or hydrogen ions) are believed to be exchanged for sodium via the Na⁺/H⁺ exchanger.

Preferably, the concentration of the compound for inhibiting transport of sodium and hydrogen across the membrane of the cell in the tissue is between about 1.0 nM to 1.0 mM. More preferably, the concentration of the compound for inhibiting transport of sodium and hydrogen across the membrane of the cell in the tissue is about 20 uM.

The inventor has also found that the inclusion of antioxidant with a potassium channel opener or adenosine receptor agonist and a compound for inducing local anaesthesia permits the viability of explanted tissue to be maintained for up to 15 hours. This is a surprising result because it has been observed by some that adenosine and lignocaine permit the function of explanted or otherwise isolated tissue to be protected for no longer than about 4 to 5 hours.

Thus in another aspect, the invention provides a composition for controlling viability of a tissue including:
a potassium channel opener or adenosine receptor agonist;
a compound for inducing local anaesthesia; and
an antioxidant.

Antioxidants are commonly enzymes or other organic substances that are capable of counteracting the damaging effects of oxidation in the tissue. The antioxidant component of the composition according to the present invention may be selected from one or more of the group consisting of: allopurinol, carnosine, Coenzyme Q 10, n-acetyl-cysteine, superoxide dismutase (SOD), glutathione reductase (GR), glutathione peroxidase (GP), catalase and the other metalloenzymes, glutathione, U-74006F, vitamin E, Trolox (soluble form of vitamin E), Vitamin C, Beta-Carotene (plant form of vitamin A), selenium, Gamma Linoleic Acid (GLA), alpha-lipoic acid, uric acid (urate), curcumin, bilirubin, proanthocyanidins, epigallocatechin gallate, Lutein, lycopene, bioflavonoids, polyphenols, trolox(R), dimethylthiourea, tempol(R), tocopherol, ascorbic acid, carotenoids, coenzyme Q, melatonin, flavonoids, polyphenols, aminoindoles, probucol and nitecapone, 21-aminosteroids or lazaroids, sulphydryl-containing compounds (thiazolidine, Ebselen, dithiolethiones). Other antioxidants include the ACE inhibitors (captopril, enalapril, lisinopril) which are used for the treatment of arterial hypertension and cardiac failure on patients with myocardial infarction. ACE inhibitors exert their beneficial effects on the reoxygenated myocardium by scavenging reactive oxygen species. Other antioxidants that could also be used include beta-mercaptopropionylglycine, 0-phenanthroline, dithiocarbamate, selegilize and desferrioxamine (Desferal), an iron chelator, has been used in experimental infarction models, where it exerted some level of antioxidant protection. Spin trapping agents such as 5′-5-dimethyl-1-pyrrolione-N-oxide (DMPO) and (a-4-pyridyl-1-oxide)-N-t-butylnitrone (POBN) also act as antioxidants.

Preferably, the antioxidant is allopurinol (1H-Pyrazolo[3,4-α]pyrimidine-4-ol). Allopurinol is a competitive inhibitor of the reactive oxygen species generating enzyme xanthine oxidase. Allopurinol's antioxidative properties may help preserve myocardial and endothelial functions by reducing oxidative stress, mitochondrial damage, apoptosis and cell death.

Preferably, the concentration of the antioxidant is between about 1 nM to 100 uM.

The inventor has also found that the inclusion of particular amounts of calcium and magnesium ions with a potassium channel opener or adenosine receptor agonist and a compound for inducing local anaesthesia permits the viability of explanted tissue to be maintained for up to 15 hours. This is a surprising result because it has been observed by some that adenosine and lignocaine permit the function of explanted or otherwise isolated tissue to be protected for no longer than about 4 to 5 hours. The effect of the particular amounts of calcium and magnesium ions is to control the amount of ions within the intracellular environment. Calcium ions tend to be depleted, exported or otherwise removed from the intracellular environment and magnesium ions tend to be increased or otherwise restored to the levels typically found in a viable, functioning cell.

Thus in another aspect, the invention provides a composition for controlling viability of a tissue including:
a potassium channel opener or adenosine receptor agonist;
a compound for inducing local anaesthesia;
a source of magnesium in an amount for increasing the amount of magnesium in a cell in the tissue; and
a source of calcium in an amount for decreasing the amount of calcium within a cell in the tissue.

As described in the present invention, elevated magnesium and low calcium has been associated with protection during ischemia and reoxygenation of the organ. The action is believed to be due to decreased calcium loading.

Preferably the magnesium is present at a concentration of between 0.5 mM to 20 mM, more preferably about 2.5 mM. Preferably the calcium present is at a concentration of between 0.1 mM to 2.5 mM, more preferably about 0.3 mM.

The composition of the invention includes at least one potassium channel opener. Potassium channel openers are agents which positively act on the channel to open it. This results in efflux of potassium across the membrane out of the cell of the tissue. It will be appreciated that the potassium channel openers include the potassium channel agonists which also stimulate the activity of the potassium channel with the same result.

The potassium channel openers may be selected from the group consisting of: nicorandil, diazoxide, minoxidil, pinicadil, aprikalim, cromokulim, emakalim, NIP121, R0316930, RWJ29009, SDZPCO400, rimakalim, symakalim, NS8, NS1608, NS1619 (1,3-dihydro-1-[2-hydroxy5(trifluoromethyl)phenyl]5-(trifluoromethyl)2-H-benimidazol-1), NS004, BMS-204352, retigabine (also GABA agonist), YM-099, YM-934, U89232 (BMS 189365), P1075, ZM244085, ZD6169, ZD0947, WAY133537 amlodipine, Bay K 8644(L-type)(1,4-dihydro-26-dimethyl-5-nitro-4[2(trifluoromethyl)phenyl]-3-pyridine carboxylic acid (methyl ester)), bepridil HCl (L-type), calciseptine (L-type), omega-conotoxin GVIA (N-type), omega-conotoxin MVIIC (Q-type), cyproheptadine HCl, dantrolene sodium (Ca²⁺ release inhibitor), diltiazem HCl (L-type), filodipine, flunarizine HCl (Ca²⁺/Na⁺), fluspirilene (L-type), HA-1077 2HCl(1-(5 isoquinolinyl sulphonyl) homo piperazine.HCl), isradipine, loperamide HCl, manoalide (Ca²⁺ release inhibitor), nicardipine HCl (L-type), nifedipine (L-type), niguldipine HCl (L-type), nimodipine (L-type), nitrendipine (L-type), pimozide (L- and T-type), ruthenium red, ryanodine (SR channels), taicatoxin, verapamil HCl (L-type), methoxy-verapamil HCl (L-type), YS-035 HCl (L-type)N[2(3,4-dimethoxyphenyl)ethyl]-3,4-dimethoxy N-nethyl benzene ethaneamine HCl) and AV blockers such as verapamil and adenosine. It will be appreciated that this list includes calcium antagonists as potassium channel openers are indirect calcium antagonists.

Adenosine (6-amino-9-β-D-ribofuranosyl-9H-purine) is particularly preferred as the potassium channel opener. Adenosine is capable of opening the potassium channel, hyperpolarising the cell, depressing metabolic function, possibly protecting endothelial cells, enhancing preconditioning of tissue and protecting from ischaemia or damage. Adenosine is also an indirect calcium antagonist, vasodilator, antiarrhythmic, antiadrenergic, free radical scavenger, arresting agent, anti-inflammatory agent (attenuates neutrophil activation), metabolic agent and possible nitric oxide donor.

Suitable adenosine receptor agonists may be selected from: N⁶-cyclopentyladenosine (CPA), N-ethylcarboxamido adenosine (NECA), 2-[p-(2-carboxyethyl)phenethyl-amino-5′-N-ethylcarboxamido adenosine (CGS-21680), 2-chloroadenosine, N⁶-[2-(3,5-demethoxyphenyl)-2-(2-methoxyphenyl]ethyladenosine, 2-chloro-N⁶-cyclopentyladenosine (CCPA), N-(4-aminobenzyl)-9-[5-(methylcarbonyl)-beta-D-robofuranosyl]-adenine (AB-MECA), ([1S-[1 a,2b,3b,4a(S*)]]-4-[7-[[2-(3-chloro-2-thienyl)-1-methyl-propyl]amino]-3H-imidazole[4,5-b]pyridyl-3-yl]cyclopentane carboxamide (AMP579), N⁶-(R)-phenylisopropyladenosine (R-PLA), aminophenylethyladenosine 9APNEA) and cyclohexyladenosine (CHA). Others include full adenosine A1 receptor agonists such as N-[3-(R)-tetrahydrofuranyl]-6-aminopurine riboside (CVT-510), or partial agonists such as CVT-2759 and allosteric enhancers such as PD81723. Other agonists may include N6-cyclopentyl-2-(3-phenylaminocarbonyltriazene-1-yl) adenosine (TCPA), a very selective agonist with high affinity for the human adenosine A1 receptor and allosteric enhancers of A1 adenosine receptor includes the 2-amino-3-napthoylthiophenes.

The composition also comprises a compound for inducing local anaesthesia, otherwise known as a local anaesthetic. The local anaesthetic component of the pharmaceutical composition according to the present invention may be selected from mexiletine, diphenylhydantoin prilocalne, procaine, mepivacaine and Class 1B antiarrhythmic agents such as lignocaine or derivatives thereof, for example, QX-314.

Preferably the local anaesthetic is Lignocaine. In this specification, the terms “lidocaine” and “lignocaine” are used interchangeably. Lignocaine is preferred as it is capable of acting as a local anaesthetic probably by blocking sodium fast channels, depressing metabolic function, lowering free cytosolic calcium, protecting against enzyme release from cells, possibly protecting endothelial cells and protecting against myofilament damage. At lower therapeutic concentrations lidocaine normally has little effect on atrial tissue, and therefore is ineffective in treating atrial fibrillation, atrial flutter, and supraventricular tachycardias. Lignocaine is also a free radical scavenger, an antiarrhythmic and has anti-inflammatory and anti-hypercoagulable properties. It must also be appreciated that at non-anaesthetic therapeutic concentrations, local anaesthetics like lidocaine would not completely block the voltage-dependent sodium fast channels, but would down-regulate channel activity and reduce sodium entry. As anti-arrhythmic, lidocaine is believed to target small sodium currents that normally continue through phase 2 of the action potential and consequently shortens the action potential and the refractory period.

As lignocaine acts by primarily blocking sodium fast channels, it will be appreciated that other sodium channel blockers could be used instead of or in combination with the local anaesthetic in the method and composition of the present invention. Examples of suitable sodium channel blockers include venoms such as tetrodotoxinand the drugs primaquine, QX, HNS-32 (CAS Registry # 186086-10-2), NS-7, kappa-opioid receptor agonist U50 488, crobenetine, pilsicainide, phenyloin, tocainide, mexiletine, NW-1029 (a benzylamino propanamide derivative), RS100642, riluzole, carbamazepine, flecainide, propafenone, amiodarone, sotalol, imipramine and moricizine, or any of derivatives thereof.

The composition according to the present invention is highly beneficial at about 10° C. but can also arrest preserve and protect over a wider temperature range up to about 37° C. In contrast, the majority of present day arrest and preservation solutions operate more effectively at lower temperatures the longer arrest times using St Thomas No. 2 solution may only be achieved when the temperature is lowered, for example, to a maximum of 4° C.

As described herein, in particular embodiments of the invention, the composition of the present invention protects and preserves tissue after arrest of the tissue, particularly after long-term arrest, with good to excellent recoveries of function or viability of the tissue after reperfusion.

Controlling viability of a tissue relates to the protection, preservation and recovery of the tissue, such that the tissue remains viable or living during those processes such that the tissue is capable of returning to its function, particularly after the tissue has been arrested, and particularly after the tissue has been arrested for over 4-6 hours. Most known solutions only provide that the tissue can be viable after shorter term arrest of up to 4-6 hours. Viability of the tissue is improved by use of the composition according to this invention.

Preservation is known as the act or process of preserving the tissue or keeping from injury, destruction or decay. In this application, the composition according to the invention acts to minimise any potential injury, destruction or decay of the tissue which may be caused by the ischemia.

Injury can be broadly characterised as reversible and irreversible cell injury. Reversible cell injury can lead to heart dysfunction usually from arrhythmias and/or stunning which is normally defined as loss of left pump function. If severe, it can lead to the death of the heart (even though the heart cells themselves are not initially dead). Irreversible injury by definition arises from actual cell death which may be fatal depending upon the extent of the injury. The amount of cell death is measured as infarct size. During recovery from cardioplegic arrest, if the conditions are adequate, the heart can be restored substantially to normal function of the tissue by reperfusion, with minimal infarct size. The most common ways to assess return of function are by measuring pressures that the heart can generate:
heart pump flow; and
the electrical activity of the heart.
   This data is then compared to data measured from pre-arrest conditions.

The term “tissue” is used herein in its broadest sense and refers to any part of the body exercising a specific function including organs and cells or parts thereof, for example, cell lines or organelle preparations. Other examples include circulatory organs such as the heart and vasculature, respiratory organs such as the lungs, urinary organs such as the kidneys or bladder, digestive organs such as the stomach, liver, pancreas or spleen, reproductive organs such as the scrotum, testis, ovaries or uterus, neurological organs such as the brain, germ cells such as spermatozoa or ovum and somatic cells such as skin cells, heart cells ie, myocytes, nerve cells, brain cells or kidney cells. The tissues may come from human or animal donors. The donor organs may also be suitable for xenotransplantation.

The composition of the present invention is particularly useful in controlling viability of heart tissue during open-heart surgery, including heart transplants, and neonate/infant hearts. Other applications include reducing heart damage before, during or following cardiovascular intervention which may include a heart attack, angioplasty or angiography. For example, the composition could be administered to subjects who have suffered or are developing a heart attack and used at the time of administration of blood clot-busting drugs such as streptokinase. As the clot is dissolved, the presence of the composition may protect the heart from further injury such as reperfusion injury. The composition may be particularly effective as a cardioprotectant in those portions of the heart that have been starved of normal flow, nutrients and/or oxygen for different periods of time. For example, the pharmaceutical composition may be used to treat heart ischaemia which could be pre-existing or induced by cardiovascular intervention.

The composition may be infused or administered as a bolus intravenous, intracoronary or any other suitable delivery route as pre-treatment for protection during a cardiac intervention such as open heart surgery (on-pump and off-pump), angioplasty (balloon and with stents or other vessel devices) and as with clot-busters (ant-clotting drug or agents).

In a preferred embodiment of this aspect of the present invention it is preferred to aerate the composition with a source of oxygen before and/or during use. The source of oxygen may be an oxygen gas mixture where oxygen is the predominant component. The oxygen may be mixed with, for example CO₂. Preferably, the oxygen gas mixture is 95% O₂ and 5% CO₂.

It is considered that the oxygenation with the oxygen gas mixture maintains mitochondrial oxidation and this helps preserve the myocyte and endothelium of the organ.

In another aspect of the present invention there is provided a method for controlling viability of a tissue including:
providing in a suitable container a composition according to the invention and a source of oxygen;
aerating the composition with the oxygen; and
placing the tissue in contact with the aerated composition under conditions sufficient to control viability of the tissue.

Preferably the oxygen source is an oxygen gas mixture. Preferably oxygen is the predominant component. The oxygen may be mixed with, for example CO₂. More preferably, the oxygen gas mixture is 95% O₂ and 5% CO₂.

Preferably the composition is aerated before and/or during contact with the tissue.

Preferably the composition according to this aspect of the invention is in liquid form. Liquid preparations of the pharmaceutical composition may take the form of, for example, solutions, syrups, or suspensions, or may be presented as a dry product for constitution with water or other suitable vehicle. Such liquid preparations may be prepared by conventional means with pharmaceutically acceptable additives such as suspending agents, emulsifying agents, non-aqueous vehicles, preservatives and energy sources.

In another aspect of the invention, there is provided a method of reducing heart tissue damage during a heart attack, cardioplegia or event likely to be ischaemic for a particular tissue or tissues by delivering a composition to the tissue, the composition according to the composition of the invention together with a suitable carrier or excipient, such as for example physiological saline or blood.

In another aspect of the invention, there is provided a method of protecting heart tissue from reperfusion injury, including inflammatory and blood coagulation effects often experienced during reperfusion following an ischaemic event. The method comprises administering a solution comprising the composition according to the present invention.

The invention also provides a method for reducing infarction size and/or reducing inflammation and blood coagulation responses in heart tissue during ischaemia and/or reperfusion comprising administration of the same solution.

The invention also provides a method for reducing electrical disturbances in the heart such as atrial or ventricular arrhythmias (including lethal ventricular tachycardias and ventricular fibrillation) during ischaemia and/or reperfusion comprising administration of the same solution.

The present invention is particularly advantageous in controlling viability of a tissue while intact in the body of a subject, for example in the treatment of the heart in circumstances of myocardial infarction or heart attack, or during surgical procedures, for example during open-heart surgery. It will also be appreciated that the present invention may also be used to control the viability of isolated tissue, such as donor organs removed from a donor.

The subject from which viability of the tissue is to be controlled may be a human or an animal such as a livestock animal (eg, sheep, cow or horse), laboratory test animal (eg, mouse, rabbit or guinea pig) or a companion animal (eg, dog or cat), particularly an animal of economic importance.

The method of the present invention involves contacting a tissue with the composition according to the invention, for a time and under conditions sufficient for the tissue to be arrested, protected and/or preserved.

While it is possible for each component of the composition to contact the tissue alone, it is preferable that the components of the pharmaceutical composition be provided together with one or more pharmaceutically acceptable carriers, diluents, adjuvants and/or excipients. Each carrier, diluent, adjuvant and/or excipient must be pharmaceutically acceptable such that they are compatible with the components of the pharmaceutical composition and not harmful to the subject. Preferably, the pharmaceutical composition is prepared with liquid carriers, diluents, adjuvants and/or excipients.

Accordingly, this aspect of the invention also provides a method for controlling viability of a tissue, which includes providing the composition together with a pharmaceutically acceptable carrier, diluent, adjuvant and/or excipient.

A preferred pharmaceutically acceptable carrier is a buffer having a pH of about 6 to about 9, preferably about 7, more preferably about 7.4 and/or low concentrations of potassium, for example, up to about 10 mM, more preferably about 2 to about 8 mM, most preferably about 4 to about 6 mM. Suitable buffers include Krebs-Henseleit which generally contains 10 mM glucose, 117 mM NaCl, 5.9 mM KCl, 25 mM NaHCO₃, 1.2 mM NaH₂PO₄, 1.12 mMCaCl₂ (free Ca²⁺=1.07 mM) and 0.512 mM MgCl₂ (free Mg²⁺=0.5 mM), Tyrodes solution which generally contains 10 mM glucose, 126 mM NaCl, 5.4 mM KCl, 1 mM CaCl₂, 1 mM MgCl₂, 0.33 mM NaH₂PO₄ and 10 mM HEPES (N-[2-hydroxyethyl]piperazine-N′-[2-ethane sulphonic acid], Fremes solution, Hartmanns solution which generally contains 129 NaCl, 5 mM KCl, 2 mM CaCl₂ and 29 mM lactate and Ringers-Lactate. One advantage of using low potassium is that it renders the present composition less injurious to the subject, in particular pediatric subjects such as neonates/infants. High potassium has been linked to an accumulation of calcium which may be associated with irregular heart beats during recovery, heart damage and cell swelling. Neonates/infants are even more susceptible than adults to high potassium damage during cardiac arrest. After surgery a neonate/infant's heart may not return to normal for many days, sometimes requiring intensive therapy or life support. It is also advantageous to use carriers having low concentrations of magnesium, such as, for example up to about 2.5 mM, but it will be appreciated that high concentrations of magnesium, for example up to about 20 mM, can be used if desired without substantially affecting the activity of the composition.

In another embodiment of the present invention there is provided use of a composition according to the present invention for controlling viability of a tissue.

Preferably the composition is aerated before and/or during contact with the tissue.

In another preferred embodiment of the present invention there is also provided a reperfusion solution which is administered after arrest and particularly after long-term arrest, together with the composition according to the invention.

Preferably, the reperfusion solution comprises Krebs Henseleit buffer.

Preferably, the reperfusion solution is provided at 37° C.

Preferably, the reperfusion solution further includes an energy substrate.

The energy substrate helps with recovering metabolism. The energy substrate can be selected from one or more components selected from the group consisting of: pyruvate, glutamate, aspartate, arginine, lactate, glucose, insulin, alpha-keto glutarate, malate, succinate, carnitine.

The invention will now be described with reference to the following examples. These examples are not to be construed as limiting the invention described in this specification in any way.

### EXAMPLES

Materials and Methods

Animals and Surgical Procedures: Male Sprague-Dawley rats weighing 300-400 g were obtained from Monash University and housed in the animal facility at James Cook University. Animals will have continual access to food and water. Rats anaesthetised with an intraperitoneal injection of sodium pentobarbital (60-70 mg/kg) and the heart excised and placed in cold Krebs Henseleit. All enzymes, chemicals and compounds were obtained from Sigma or Boehringer-Mannheim. Lignocaine were purchased from the local organ arrest, protection and/or preservation suppliers. Adenosine was purchased from Biomedical Res. Ltd (Sigma).

Isolated Working Rat Heart: Hearts were perfused in the working mode. Oxygen and CO₂ tensions, and pH in the arterial and venous perfusion lines were measured using a Corning 865 pH/blood gas-ion-analyser. Physiological variables were measured using a single channel Mac-Lab with a pressure transducer (UFI-1050) attached.

Preservation Solution: 200 uM adenosine plus 0.5 mM lignocaine, 10 uM diazoxide, 70 mM sucrose, 100 uM allopurinol in Krebs Henseleit (described below) and 10 mM glucose (gently aerated with 95% O₂ and 5% CO₂). The pH of the solution at 10° C. was approximately 7.3, pCO₂=53 mmHg and pO₂ around 700 mmHg O₂. Note: CaCl₂ is 0.3 mM and MgCl₂ is 2.5 mM.

Krebs-Henseleit buffer: NaCl (117 mM), KCl (5.9 mM), NaHCO₃ (25 mM), NaH₂PO₄, (1.2 mM) 1.12 mM CaCl₂ (free Ca²⁺=1.07 mM), 0.512 mM MgCl₂ (free Mg²⁺=0.5 mM), pH 7.4 at 38° C. (aerated with 95% O₂ and 5% CO₂).

Mode of delivery: The preservation solution was delivered continuously at a ‘constant perfusion’ head of 30 cmH₂O with temperature maintained using a refrigerated water-bath. It was gently aerated with 95% O₂ 5% CO₂ taking care to avoid wide swings in pH. While it is true that cold immersion storage (4° C.) is the most popular technique for long term heart preservation (4-6 hrs), over the past 10 years many studies have demonstrated the superiority of the ‘constant perfusion’ method for heart protection and preservation. Some of the advantages of ‘continuous perfusion’ over cold immersion storage are: (1) reducing the likelihood of ischaemia, anaerobic metabolism and reperfusion injury, (2) increased supply of nutritional requirements (ie. energy substrates), and (3) removal of waste products from the coronary circulation. In summary, the available published data demonstrate that continuous perfusion improves preservation of donor hearts compared to “static” immersion cold storage. However, the present invention and methodology does not preclude the use of static storage.

### Example 1

Arrest time 15 hours

Preservation solution (as described in Materials and Methods). Preservation temperature was 10° C.

Reperfusion Solution: Oxygenated Krebs Henseleit containing 10 mM glucose, 70 mM sucrose and 1 mM pyruvate. Reperfusion temperature was at 37° C.

Results: Table 1 below summarises the effect of the new invention on the isolated rat heart after 15 hours arrest at 10° C. At 5 min the heart recovered nearly full function (87-100%).

TABLE 1

| | Heart | Systolic | Diastolic | | Coronary |
|---|---|---|---|---|---|
| | rate | Press | Press | Aortic flow | Flow |
| | (bpm) | (mmHg) | (mmHg) | (ml/min) | (ml/min) |
| Pre-arrest | 257 | 124 | 69 | 36 | 13 |
| Recovery | | | | | |
| 5 min | 224 | 132 | 69 | 32 | 13.8 |
| % of control | (87%) | (100%) | (100%) | (89%) | (101%) |

### Example 2

Arrest time 12 hours

Preservation solution (as described in Materials and Methods but with 90 mM sucrose, not 70 mM sucrose). Preservation temperature was 10° C.

Reperfusion Solution: Oxygenated Krebs Henseleit containing 10 mM glucose, 90 mM sucrose, 1.0 mM pyruvate and 1.0 mM glutathione. Reperfusion temperature was at 37° C.

Results: Table 2 below summarises the effect of the new invention on the isolated rat heart after 12 hours arrest at 10° C. At 5 min the heart rate recovered 61% of control, aortic and coronary flows about 50% and developed pressures a little over 100%.

TABLE 2

| | | Systolic | Diastolic | | Coronary |
|---|---|---|---|---|---|
| | Heart rate | Press | Press | Aortic flow | Flow |
| | (bpm) | (mmHg) | (mmHg) | (ml/min) | (ml/min) |
| Pre-arrest | 243 | 136 | 70 | 46 | 16 |
| Recovery | | | | | |
| 5 min | 148 | 142 | 74 | 25 | 8 |
| % of | 61% | 105% | 106% | 54% | 50% |
| control | | | | | |

### Example 3

Arrest time 12 hours

Preservation solution (as described in Materials and Methods but with 90 mM sucrose and no allopurinol). Preservation temperature was 10° C.

Reperfusion Solution: Oxygenated Krebs Henseleit containing 10 mM glucose, 90 mM sucrose and with no allopurinol and no pyruvate. Reperfusion temperature was at 37° C.

Results: Table 3 below summarises the effect of the new invention on the isolated rat heart after 12 hours arrest at 10° C. At 5 min the heart rate recovered 73% of control, aortic flow 40%, coronary flow 86% and developed pressures 110% of control measured 12 hours earlier.

TABLE 3

| | Heart | Systolic | Diastolic | | Coronary |
|---|---|---|---|---|---|
| | rate | Press | Press | Aortic flow | Flow |
| | (bpm) | (mmHg) | (mmHg) | (ml/min) | (ml/min) |
| Pre-arrest | 333 | 114 | 71 | 40 | 21.5 |
| Recovery | | | | | |
| 5 min | 243 | 127 | 78 | 16.2 | 18.4 |
| % of control | 73% | 111% | 110% | 40% | 86% |

### Example 4

Arrest time 6 hours

Preservation solution (as described in Materials and Methods but with 90 mM sucrose). Preservation temperature was 10° C.

Reperfusion Solution: Oxygenated Krebs Henseleit containing 10 mM glucose, 90 mM sucrose and 20 uM amiloride. No allopurinol and or pyruvate. Reperfusion temperature was at 37° C.

Results: Table 4 below summarises the effect of the new invention on the isolated rat heart after 12 hours arrest at 10° C. At 5 min the heart rate recovered 60% of control, aortic flow 63%, coronary flow 120% and developed pressures 93-118% of control measured 12 hours earlier.

TABLE 4

| | | Systolic | Diastolic | | Coronary |
|---|---|---|---|---|---|
| | Heart rate | Press | Press | Aortic flow | Flow |
| | (bpm) | (mmHg) | (mmHg) | (ml/min) | (ml/min) |
| Pre-arrest | 385 | 115 | 75 | 43.5 | 17.5 |
| Recovery | | | | | |
| 5 min | 233 | 136 | 70 | 27.5 | 21 |
| % of | 60% | 118% | 93% | 63% | 120% |
| control | | | | | |

### GENERAL CONCLUSION

The results from four examples show that the new long-term preservation solution can preserve the rat heart for up to 15 hours with good to excellent recoveries measured at five minutes after the onset of reperfusion.

1-29. (canceled)

30. A composition for controlling viability of a tissue including:
a potassium channel opener or adenosine receptor agonist;
a compound for inducing local anaesthesia; and
a compound for inhibiting transport of sodium and hydrogen ions across a plasma membrane of a cell in the tissue.

31. A composition according to claim 30 wherein the compound for inhibiting transport of sodium and hydrogen ions is selected from the group consisting of: N-amidino-3,5-diamino-6-chloropyrzine-2-carboximide hydrochloride dehydrate, EIPA, cariporide (HOE—642), eniporide, Triamterene, EMD 84021, EMD 94309, EMD 96785, EMD 85131, HOE 694, B11 B-513 and T-162559.

32. A composition according to claim 30 wherein the compound is N-amidino-3,5-diamino-6-chloropyrzine-2-carboximide hydrochloride dehydrate.

33. A composition according to claim 30 wherein the concentration of the compound is between about 1 nM to 1 mM.

34. A composition according to claim 30 wherein the composition further includes at least one compound selected from the group consisting of:
a compound for reducing the uptake of water by a cell in the tissue;
diazoxide;
a source of magnesium in an amount for increasing the amount of magnesium in a cell in the tissue; and
a source of calcium in an amount for decreasing the amount of calcium within a cell in the tissue.

35. A composition for controlling viability of a tissue including:
a potassium channel opener or adenosine receptor agonist;
a compound for inducing local anaesthesia; and
an antioxidant.

36. A composition according to claim 35 wherein the antioxidant is selected from the group consisting of: allopurinol, carnosine, Coenzyme Q 10, n-acetyl-cysteine, superoxide dismutase (SOD), glutathione reductase (GR), glutathione peoxidase (GP), catalase and the other metalloenzymes, glutathione, U-74006F, vitamin E, Trolox (soluble form of vitamin E), Vitamin C, Beta-Carotene (plant form of vitamin A), selenium, Gamma Linoleic Acid (GLA), alpha-lipoic acid, uric acid (urate), curcumin, bilirubin, proanthocyanidins, epigallocatechin gallate, Lutein, lycopene, bioflavonoids, polyphenols, trolox(R), dimethylthiourea, tempol(R), tocopherol, ascorbic acid, carotenoids, coenzyme Q, melatonin, flavonoids, polyphenols, aminoindoles, probucol, nitecapone, 21-aminosteroids, lazaroids, sulphydryl-containing compounds, ACE inhibitors, beta-mercaptopropionylglycine, 0-phenanthroline, dithiocarbamate, selegilize, desferrioxamine (Desferal), 5′-5-dimethyl-1-pyrrolione-N-oxide (DMPO) and a-4-pyridyl-1-oxide)-N-t-butylnitrone (POBN).

37. A composition according to claim 35, wherein the antioxidant is allopurinol.

38. A composition according to claim 35, wherein the concentration of the antioxidant is between about 1 nM to 100 uM.

39. A composition according to claim 35 wherein the composition further includes at least one compound selected from the group consisting of:
a compound for reducing the uptake of water by a cell in the tissue;
diazoxide;
a source of magnesium in an amount for increasing the amount of magnesium in a cell in the tissue; and
a source of calcium in an amount for decreasing the amount of calcium within a cell in the tissue.

40. A method of controlling the viability of a tissue including the step of contacting the tissue with a composition according to claim 30.

41. A method according to claim 40 wherein the composition is treated to oxygenate the composition prior to or while the composition is in contact with the tissue.

42. A method according to claim 40 wherein the tissue is contacted by continuous perfusion of the composition the composition being at a temperature of about 10° C.

43. A method of controlling the viability of a tissue including the step of contacting the tissue with a composition according to claim 35.

44. A method for arresting a tissue including the step of contacting the tissue with a composition according to claim 30.

45. A method for arresting a tissue including the step of contacting the tissue with a composition according to claim 35.

46. A method for preserving a tissue including the step of contacting the tissue with a composition according to claim 30.

47. A tissue preserved by the method according to claim 46.

48. A heart tissue preserved tissue according to claim 46.

49. A method for preserving a tissue including the step of contacting the tissue with a composition according to claim 35.
